(19) United States
(12) Patent Application Publication
(10) Pub. No.: US 2019/0168020 A1
(43) Pub. Date: Jun. 6, 2019

(54) LIGHT SOURCE APPARATUS AND WEARABLE APPARATUS

(71) Applicant: Industrial Technology Research Institute, Hsinchu (TW)

(72) Inventors: Wei-Cheng Chao, Kaohsiung City (TW); Chi-Chin Yang, Hsinchu City (TW); Li-Chi Su, Yilan County (TW); Mu-Tao Chu, Hsinchu City (TW)

(73) Assignee: Industrial Technology Research Institute, Hsinchu (TW)

(21) Appl. No.: 15/884,309

(22) Filed: Jan. 30, 2018

(30) Foreign Application Priority Data
Dec. 6, 2017 (TW) 106142696

(51) Int. Cl. A61N 5/06; A61M 21/02

(52) U.S. Cl. CPC A61N 5/0622; A61M 2021/0044; A61M 21/02

(57) ABSTRACT

A light source apparatus including a light source is provided. The light source emits a light beam. The light beam illuminates a user so that at least one brain wave index of at least one region in frontal lobe regions of the user changes. A wearable apparatus is also provided.

### CROSS-REFERENCE TO RELATED APPLICATION

This application claims the priority benefit of Taiwan application serial no. 106142696, filed on Dec. 6, 2017. The entirety of the above-mentioned patent application is hereby incorporated by reference herein and made a part of this specification.

### BACKGROUND

### Technical Field

The disclosure relates to a light source apparatus and a wearable apparatus.

### Description of Related Art

Studies show that, in addition to causing instantaneous effect on visual perceptions of people, a light beam also causes short-term or long-term impact on vision, psychology, physiological effect, and biological effect of people. In the long run, psychology, emotion, mental state, cognition, and behavior of the human body may be affected. Therefore, techniques have been proposed to improve the psychology, emotion, mental state, cognition, behavior, etc. of the human body by using light treatment. In the related art, light treatment generally involves a white light source of high intensity and is thus likely to cause side effect to the human body or cause visual discomfort. Moreover, in the light treatment of the related art, a light source modulation method, optimal light source recipes corresponding to different states, an implementation procedure, and light health care and light treatment products and systems are not designed by taking into account the impact of the light beam on the vision, psychology, physiological effect, and biological effect of the human body.

### SUMMARY

The embodiments of the disclosure provide a light source apparatus in which optimal light source recipes corresponding to different purposes of light source application are designed by taking into account vision, psychology, physiological effect, and biological effect of the human body, and effect of adjustment of an emotional state, health care, or treatment is thereby achieved.

The embodiments of the disclosure further provide a wearable apparatus using the foregoing light source apparatus.

A light source apparatus of an embodiment of the disclosure includes a light source. The light source emits a light beam. The light beam illuminates a user so that at least one brain wave index of at least one region in frontal lobe regions of the user changes.

A wearable apparatus of an embodiment of the disclosure includes the foregoing light source apparatus and a fixing member, wherein the light source apparatus is disposed on the fixing member and is located around eyes of the user.

To provide a further understanding of the aforementioned and other features and advantages of the disclosure, exemplary embodiments, together with the reference drawings, are described in detail below.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a schematic diagram illustrating a light source apparatus according to an embodiment of the disclosure.

FIG. 2 is a schematic diagram illustrating an arrangement of electrodes at the time of obtaining an electroencephalograph (EEG).

FIG. 3 is a flowchart illustrating a method for confirming a color temperature range, an illuminance range, and a color rendering index (CRI) range of a white light according to an embodiment of the disclosure.

FIG. 4 is a flowchart illustrating a method for confirming a wavelength range and an intensity range of a monochromatic light according to an embodiment of the disclosure.

FIG. 5 is a flowchart illustrating a method for modulating a light source apparatus according to an embodiment of the disclosure.

FIG. 6 and FIG. 7 are two schematic diagrams respectively illustrating a wearable apparatus according to embodiments of the disclosure.

FIG. 8 and FIG. 9 are schematic diagrams illustrating a light source apparatus according to other embodiments of the disclosure.

### DESCRIPTION OF THE EMBODIMENTS

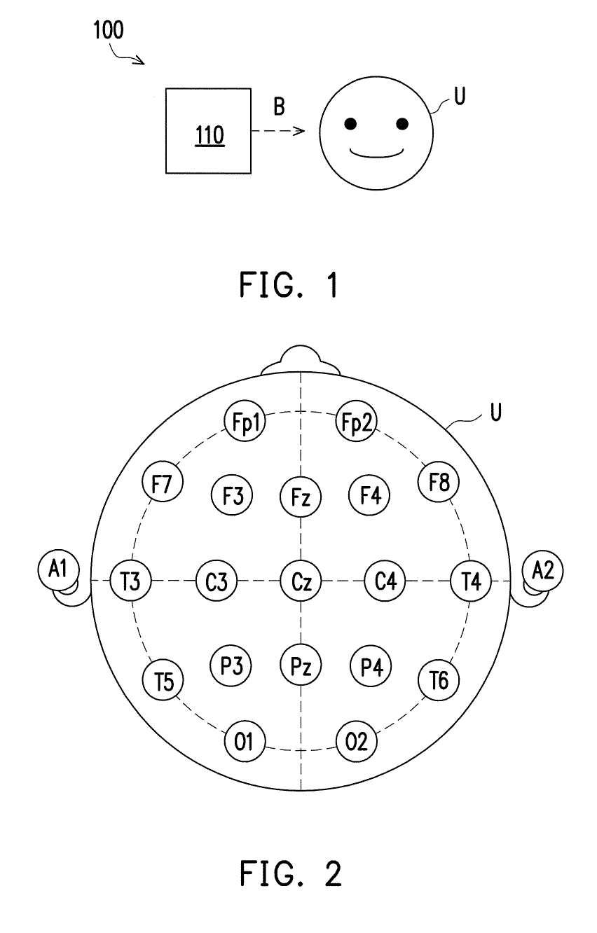

FIG. 1 is a schematic diagram illustrating a light source apparatus according to an embodiment of the disclosure. Referring to FIG. 1, a light source apparatus 100 of the embodiment includes a light source 110. The light source 110 emits a light beam B. The light beam B illuminates a user U so that at least one brain wave index of at least one region in frontal lobe regions of the user U changes.

Specifically, to learn whether at least one brain wave index of at least one region in the frontal lobe regions of the user U changes after illumination of the light beam, an electroencephalograph of the user U is obtained through an electroencephalography device. FIG. 2 is a schematic diagram illustrating an arrangement of electrodes at the time of obtaining the electroencephalograph. Generally, when an electroencephalograph is obtained, pairs of electrodes are disposed on the head and ears (in regions A1, A2) of the user U to record brain wave indexes of frontal lobe regions (including regions Fp1, Fp2, F3, F4, Fz, F7, F8), temporal lobe regions (including regions T3, T4, T5, T6), parietal lobe regions (including regions P3, P4, Pz), occipital lobe regions (including regions O1, O2), and central sulcus regions (including regions C3, C4, Cz) in the cerebral cortex of the brain. The brain wave index includes, for example, at least one of an α wave power, an α wave amplitude, a β wave amplitude, a low β wave amplitude, a γwave amplitude, a θ wave amplitude, and a ratio of the β wave amplitude and the α wave amplitude (i.e., β/α), but is not limited hereto.

Different brain wave indexes indicate different states of the human body. Therefore, by observing at least one brain wave index of at least one region in the frontal lobe regions of the user U through the electroencephalograph, it is possible to learn about a state of the user U, such as an emotional state, a mental state, and a sleep state. Here, the emotional state generally refers to visual or psychological perceptions of the user U, such as those that are relaxing, delightful, energetic, focused, awake, mild, vivid, bright, glareless, comfortable, cold, warm, or any permutation or combination of the above, but the disclosure is not limited hereto. The mental state generally refers to mental treatment issues, such as depression, seasonal affective disorder (SAD), generalized anxiety disorder (GAD), Alzheimer's disease (AD), Parkinson's disease (PD), or attention deficit hyperactivity disorder (ADHD), but the disclosure is not limited hereto. The sleep state generally refers to sleep health care issues and sleep treatment issues. The sleep health care issues include, for example, assisting in falling asleep, shortening time taken to fall asleep, improving sleep quality, etc., but are not limited hereto. The sleep treatment issues include issues such as delayed sleep phase disorder (DSPD), advanced sleep phase disorder (ASPD), shift work disorder (SWD), jet lag, or insomnia.

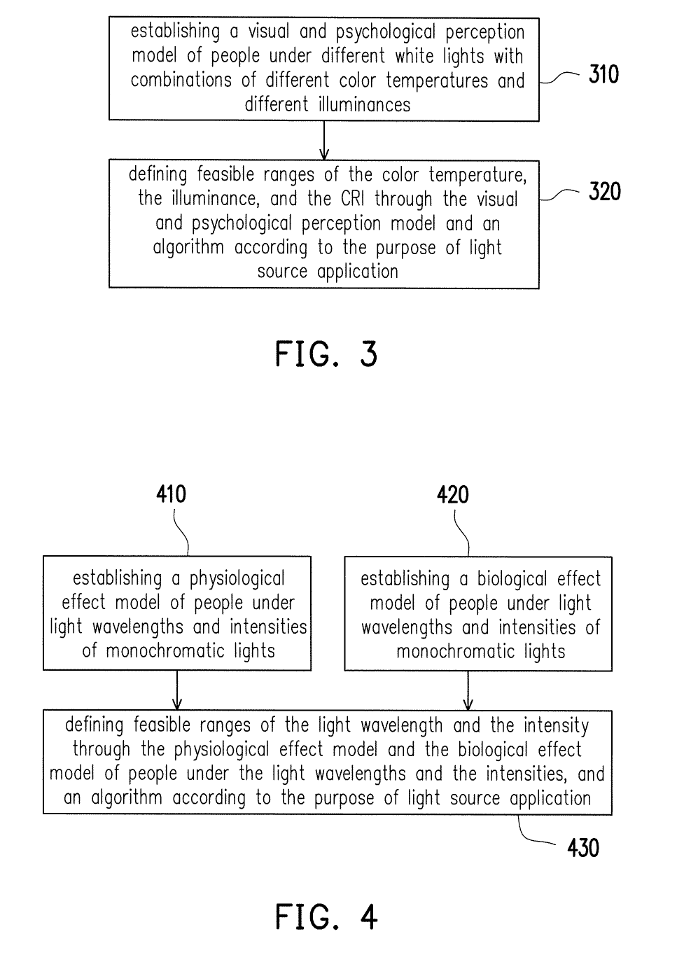

With a different purpose of light source application (e.g., adjustment of the emotional state, health care, or treatment), the light source apparatus is required to have a corresponding optimal light source recipe. One of a plurality of methods for adjusting an optimal light source recipe of a light source apparatus will be described below with reference to FIG. 3 to FIG. 5.

FIG. 3 is a flowchart illustrating a method for confirming a color temperature range, an illuminance range, and a color rendering index (CRI) range of a white light according to an embodiment of the disclosure. Referring to FIG. 3, first, in step 310, a visual and psychological perception model of people under different white lights with combinations of different color temperatures and different illuminances is established. Specifically, visual and psychological perceptions of a plurality of subjects under different color temperature-illuminance combinations are obtained through an ergonomic experiment, and the visual and psychological perception model is then constructed according to the experiment result.

In an example, the plurality of subjects in the ergonomic experiment include males and females, and ages of the subjects fall in a range of 20 to 80. Moreover, the color temperature of the white light falls in a range of 2500 K to 7000 K, and the illuminance of the white light falls in a range of 200 lux to 3000 lux. As the CRI becomes higher, a color expression becomes closer to an ideal light source or natural light, and an object is presented in more real colors under illumination of the light beam. Accordingly, in the ergonomic experiment, the CRI of the white light is set to be greater than or equal to 70. Under multiple color temperature-illuminance combinations, the subjects rate opposite visual and psychological perceptions. The visual and psychological perception model is constructed according to a rating result. Here, opposite visual and psychological perceptions include, for example, “weak and strong”, “mild and vivid”, “dark and bright”, “glare and glareless”, “tense and relaxed”, “tired and awake”, “depressing and delightful”, “uncomfortable and comfortable”, and “cold and warm”.

Next, referring to step 320, according to the purpose of light source application, feasible ranges of the color temperature, the illuminance, and the CRI are defined through the visual and psychological perception model and an algorithm. Specifically, according to a difference in the emotional state, the mental state, or the sleep state to be adjusted, the combination of the color temperature range, the illuminance range, and the CRI range of the white light is also different. In step 320, the color temperature range, the illuminance range, and the CRI range in the feasible ranges of the white light are confirmed for different purposes of light source application.

FIG. 4 is a flowchart illustrating a method for confirming a wavelength range and an intensity range of a monochromatic light according to an embodiment of the disclosure. Referring to FIG. 4, first, in step 410 and step 420, a physiological effect model and a biological effect model of people under light wavelengths and intensities of different monochromatic lights are established. Here, the physiological effect generally refers to common physiological responses, such as a body temperature, a heart rate, alertness, cognitive performance, psychomotor performance, brain blood flow, EEG responses, clock gene expression, circadian regulation, or other mental treatment issues. The biological effect generally refers to changes in hormone secretion. Here, hormone includes, for example, at least one of cortisol, endorphin, oxytocin, dopamine, serotonin, γ-aminobutyric acid (GABA), acetylcholine, melatonin, leptin, and norepinephrine/noradrenaline, but the disclosure is not limited hereto.

Physiological responses and changes in hormone secretion of a plurality of subjects under light wavelengths and intensities of different monochromatic lights are obtained through an ergonomic experiment, and the physiological effect model and the biological effect model are then constructed according to the experiment result. For example, the physiological effect on the subjects may be confirmed by observing changes in the brain wave index in the electroencephalograph, by analyzing a heart rate variability (HRV), or by analyzing a galvanic skin response (GSR), and the physiological effect model may be constructed according to the test result. Moreover, the biological effect on the subjects may be confirmed by observing changes in hormone secretion in the human body, and the biological effect model may be constructed according to the test result.

In an example, the plurality of subjects in the ergonomic experiment include males and females, and ages of the subjects fall in a range of 20 to 80. Moreover, the wavelength of the monochromatic light falls in a range of 380 nm to 780 nm, and an irradiance (intensity) of the monochromatic light entering the eyes of the user falls in a range of 30 μW/cm² to 200 μW/cm². In addition, in the ergonomic experiment, light illumination time points of the user are distributed within 24 hours. A frequency of light illumination ranges from three times a day to once a week. A duration of light illumination each time is 0.5 hours to 4 hours. A total course of light illumination falls in a range of 1 day to 3 months.

Next, referring to step 430, according to the purpose of light source application, feasible ranges of the light wavelength and the intensity are defined through the physiological effect model and the biological effect model of people under the light wavelengths and the intensities, and an algorithm. Specifically, according to a difference in the emotional state, the mental state, or the sleep state to be adjusted, the combination of the wavelength range and the intensity range of the monochromatic light is also different. In step 430, the wavelength range and the intensity range in the feasible ranges of the monochromatic light are configured for different purposes of light source application.

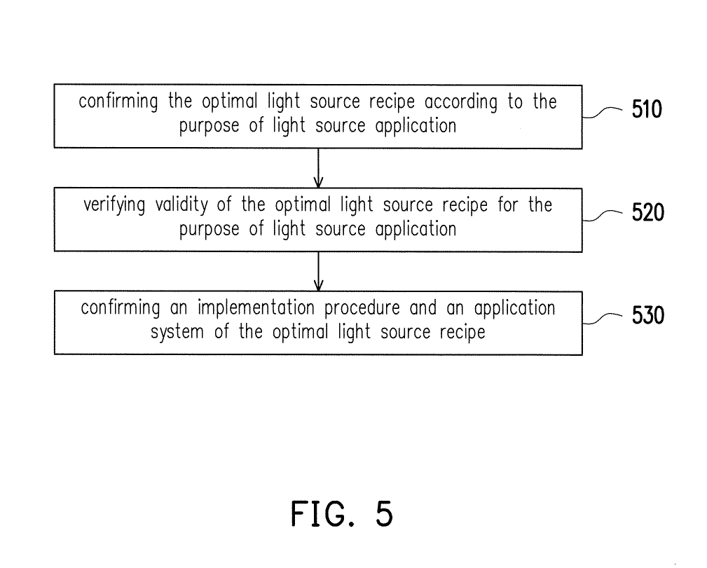

FIG. 5 is a flowchart illustrating a method for modulating a light source apparatus according to an embodiment of the disclosure. Referring to FIG. 5, first, in step 510, according to the purpose of light source application, the optimal light source recipe is confirmed. Specifically, through the steps shown in FIG. 3, it is learned how to modulate the recipe (including the color temperature range, the illuminance range, and the CRI range) of the white light to cause the user to have specific visual and psychological perceptions. Through the steps shown in FIG. 4, it is learned how to modulate the recipe (including the light wavelength range and the intensity range) of the monochromatic light to cause specific physiological responses or hormone secretion in the user. When the foregoing two results are integrated, vision, psychology, physiological effect, and biological effect of the human body are comprehensively considered, and namely, the color temperature, the illuminance, and the CRI of the light beam, and the irradiance (intensity) of different color lights (different wavelengths) in the light beam are controlled based on specific purposes of light source application.

Next, referring to step 520, validity of the optimal light source recipe for the purpose of light source application is verified. For example, by measuring whether the brain wave index of a specific region in the frontal lobe regions of the user changes, and whether a p-value of a statistical test on the change is smaller than 0.05 (meaning that the change in at least one brain wave index after light illumination is significant), it is determined whether the emotional state, the mental state, or the sleep state of the user is actually changed.

In an embodiment, when the color temperature of the light beam falls in a range of 4500 K to 6500 K, the illuminance of the light beam falls in a range of 700 lux to 3000 lux, the CRI of the light beam is greater than or equal to 70, and the irradiance of a blue light in the light beam entering the eyes of the user falls in a range of 30 μW/cm² to 200 μW/cm², a delightful perception is generated in the user. Upon verification, under illumination of the light beam adopting the foregoing optimal light source recipe, secretion of serotonin in the user increases. Moreover, in the frontal lobe regions, the region F4, the region F8, the region Fp2 (see FIG. 2, hereinafter collectively referred to as a first region), and the region F3, the region F7, and the region Fp1 (see FIG. 2, hereinafter collectively referred to as a second region) satisfy: Ln(F4)−Ln(F3)>0, Ln(F8)−Ln(F7)>0, or Ln(Fp2)−Ln(Fp1)>0, wherein Ln(F4) is a natural logarithm of the α wave or θ wave power of the first region in the frontal lobe regions of the user, Ln(F3) is a natural logarithm of the α wave or θ wave power of the second region in the frontal lobe regions of the user, Ln(F8) is a natural logarithm of the α wave or θ wave power of the first region in the frontal lobe regions of the user, Ln(F7) is a natural logarithm of the α wave or θ wave power of the second region in the frontal lobe regions of the user, Ln(Fp2) is a natural logarithm of the α wave or θ wave power of the first region in the frontal lobe regions of the user, and Ln(Fp1) is a natural logarithm of the α wave or θ wave power of the second region in the frontal lobe regions of the user. The phenomenon above shows that positive emotion is generated in the user after light illumination.

In another embodiment, when the color temperature of the light beam falls in a range of 4500 K to 6500 K, the illuminance of the light beam falls in a range of 700 lux to 3000 lux, the CRI of the light beam is greater than or equal to 70, the irradiances of a blue light and a green light in the light beam entering the eyes of the user respectively fall in a range of 30 μW/cm² to 200 μW/cm², and the irradiance of the green light is greater than the irradiance of the blue light, an awakening perception is generated in the user. Upon verification, under illumination of the light beam adopting the foregoing optimal light source recipe, a ratio (i.e., β/α) of the β wave amplitude and the α wave amplitude and the γwave amplitude of the regions Fp1, Fp2, F3, F4, Fz (see FIG. 2) in the frontal lobe regions of the user are effectively increased. The phenomenon above shows that the user feels more awake and concentrated after light illumination.

In still another embodiment, when the color temperature of the light beam falls in a range of 3000 K to 4500 K, the illuminance of the light beam falls in a range of 400 lux to 800 lux, the CRI of the light beam is greater than or equal to 70, and the irradiance of a green light in the light beam entering the eyes of the user falls in a range of 30 μV/cm² to 200 μW/cm², a relaxing perception is generated in the user. Upon verification, under illumination of the light beam adopting the foregoing optimal light source recipe, the low β wave amplitude and the θ wave amplitude of the regions Fp1, Fp2, F3, F4, Fz (see FIG. 2) in the frontal lobe regions of the user are effectively increased. Moreover, the α wave amplitude of the regions F3, F4, Fz (see FIG. 2) in the frontal lobe regions of the user is effectively increased. The phenomenon above shows that the user feels more relaxed after light illumination.

In yet another embodiment, when the color temperature of the light beam falls in a range of 3000 K to 4500 K, the illuminance of the light beam is smaller than or equal to 600 lux, the CRI of the light beam is greater than or equal to 70, and the irradiance of a green light in the light beam entering the eyes of the user falls in a range of 30 μW/cm² to 200 μW/cm², sleepiness in the user is enhanced. Upon verification, under illumination of the light beam adopting the foregoing optimal light source recipe, the θ wave amplitude in the frontal lobe regions, the occipital lobe regions, and the parietal lobe regions of the user are effectively increased. Moreover, the α wave amplitude in the occipital lobe regions of the user is effectively decreased. The phenomenon above shows that the user feels sleepy after light illumination.

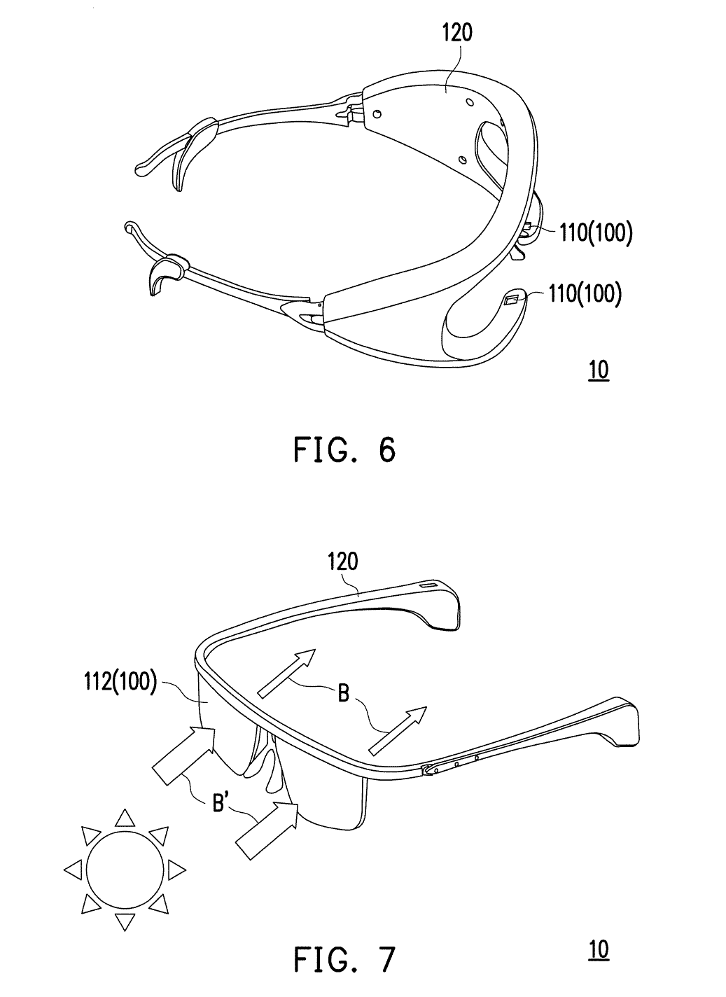

Then, referring to step 530, an implementation procedure and an application system of the optimal light source recipe are confirmed. The implementation procedure includes, for example, at least one of the light illumination time points, the duration of light illumination each time, the frequency of light illumination, and the total course of light illumination. The application system of the optimal light source recipe refers to a specific mode of implementation of the light source apparatus. Two specific modes of implementation of the light source apparatus are described below with reference to FIG. 6 and FIG. 7, but the possible modes of implementation of the light source apparatus are not limited to those illustrated in FIG. 6 and FIG. 7.

FIG. 6 and FIG. 7 are two schematic diagrams respectively illustrating a wearable apparatus according to embodiments of the disclosure. Referring to FIG. 6, a wearable apparatus 10 includes a light source apparatus 100 and a fixing member 120, wherein the light source apparatus 100 is mounted around the eyes of a user through the fixing member 120. For example, the fixing member 120 may be in a form of glasses, and a light source 110 of the light source apparatus 100 may be disposed in the fixing member 120 at a position close to the nasal bridge or at another position close to the eyes. However, the form of the fixing member 120, the configuration position of the light source 110, and the number of light sources may be changed according to the requirement and are not limited to those illustrated in FIG. 6. For example, the fixing member 120 may also be in forms of goggles, a helmet, a head covering, or another form, and the light source 110 may be disposed at any position in the fixing member 120 without affecting view of the user.

In the embodiment, the light source 110 includes at least one red light emitting element, at least one green light emitting element, and at least one blue light emitting element to mix and produce the required white light. The light emitting element is, for example, a light emitting diode, but is not limited hereto. By adjusting the color temperature range, the illuminance range, and the CRI range of the white light, specific visual and psychological perceptions are generated in the user. Moreover, by adjusting the intensity of specific monochromatic lights (e.g., at least one of a green light and a blue light) in the white light, specific physiological responses or hormone secretion is caused in the user, and effect of adjustment of the emotional state, health care, or treatment is thereby achieved. In an embodiment, the light source 110 may also be a monochromatic light emitting element with phosphor powders or a monochromatic light emitting element with quantum dots that mixes to produce a white light. Alternatively, the light source 110 may also be a white light source of another type functioning with an optical filter module (including an optical filter) to adjust the color temperature range, the illuminance range, and the CRI range of the white light. Alternatively, as shown in FIG. 7, the light source in the light source apparatus 100 may be an optical filter module 112 instead of a light emitting element. Specifically, the light source apparatus 100 uses an ambient light or a sunlight B′ as the light source, and the optical filter module 112 adjusts the color temperature range, the illuminance range, the CRI range of the light beam B in the ambient light or sunlight illuminated on the user, and the intensity of specific monochromatic lights (e.g., at least one of the green light and the blue light) in the white light, thereby achieve the foregoing effect of adjustment of the emotional state, health care, or treatment.

In the framework where the fixing member 120 is disposed, the light source 110 is mounted around the eyes of the user, and the light beam emitted by the light source 110 is configured not to significantly affect the view of the user, such that the impact caused by the light beam emitted by the light source 110 on the vision and psychology of people is reduced. Accordingly, the physiological effect and the biological effect caused by the light beam on the user may be simply considered. In that case, it is possible that the light source 110 does not provide a white light. For example, it is possible that the light emitting elements in the light source 110 do not include the red light emitting element, but only include at least one of the green light emitting element and the blue light emitting element.

On the other hand, if the light source apparatus 100 is constructed in a form of a light fixture (e.g., a fluorescent lamp or a table lamp) providing a larger range of illumination, then the light source apparatus 100 preferably provides a white light to reduce disturbance caused by the light treatment or light health care to the user or to other people in the same space. Specifically, if the light source apparatus 100 is constructed in the form of a light fixture providing a larger range of illumination, then the light source apparatus 100 preferably provides a white light, and the white light is adjusted to the optimal light source recipe (the color temperature range, the illuminance range, the CRI range, and the irradiance range of different color lights in the white light) according to the purpose of light source application. Accordingly, while adjustment of the emotional state, health care, or treatment is achieved, discomfort or disturbance caused to the user or other people in the same space is reduced to an acceptable or imperceptible degree.

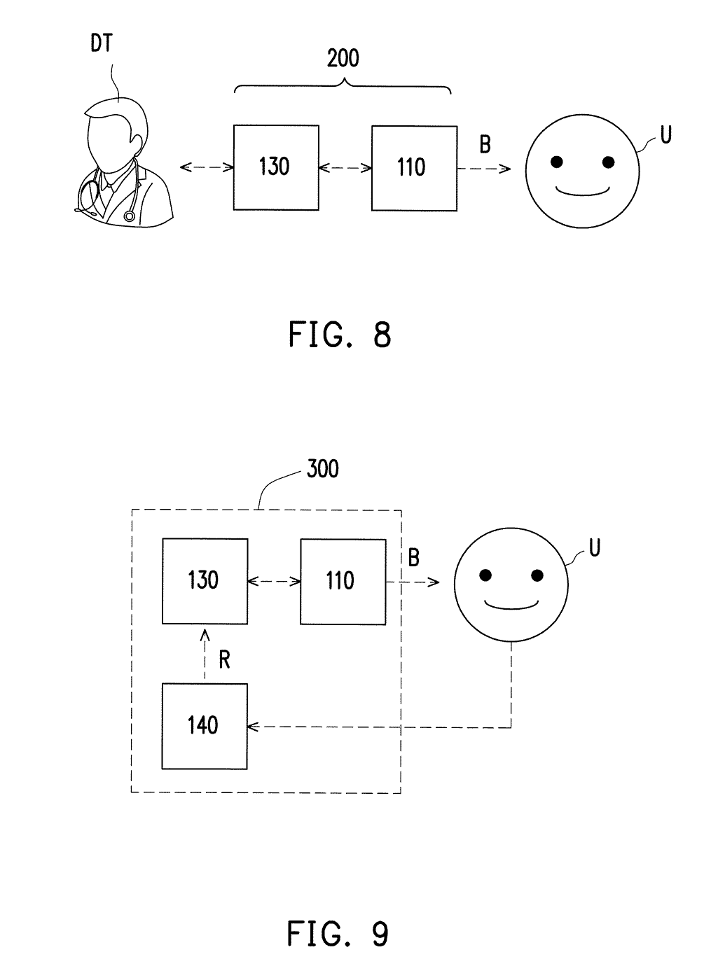

FIG. 8 and FIG. 9 are schematic diagrams illustrating a light source apparatus according to other embodiments of the disclosure. Referring to FIG. 8, a light source apparatus 200 of the embodiment is similar to the light source apparatus 100 of FIG. 1, wherein the same components are labeled with the same numerals and will not be repeatedly described below. A main difference between the light source apparatus 200 and the light source apparatus 100 is described below. In the light source apparatus 200, the light source apparatus 200 further includes a controller 130. The controller 130 is coupled to the light source 110, and the controller 130 is adapted to change at least one of the color temperature, the illuminance, the CRI of the light beam B emitted by the light source 110, the irradiances of different color lights in the light beam B, the light illumination time points, the duration of light illumination each time, the frequency of light illumination, and the total course of light illumination of the user U.

For example, the controller 130 sets at least one of the color temperature, the illuminance, the CRI of the light beam B, the irradiances of different color lights in the light beam B, the light illumination time points, the duration of light illumination each time, the frequency of light illumination, and the total course of light illumination of the user U according to a diagnosis of a doctor DT. In the framework where the light source apparatus 200 is a portable light source apparatus, the user U may undergo light treatment or light health care wherever he or she is. On the other hand, if the light source apparatus 200 is in the form of a fixed light fixture, it is up to the user U to select the location for placing the light source apparatus 200, and it is possible to undergo light treatment or light health care without going to a clinic or hospital.

Referring to FIG. 9, a light source apparatus 300 of the embodiment is similar to the light source apparatus 200 of FIG. 8, wherein the same components are labeled with the same numerals and will not be repeatedly described below. A main difference between the light source apparatus 300 and the light source apparatus 200 is described below. In the light source apparatus 300, the light source apparatus 300 further includes a physiological monitoring apparatus 140. The physiological monitoring apparatus 140 is adapted to monitor a state of the user U, and the physiological monitoring apparatus 140 is coupled to the controller 130 to transmit a measurement result R to the controller 130 in a wired or wireless manner. The controller 130 changes at least one of the color temperature, the illuminance, the CRI of the light beam B, the irradiances of different color lights in the light beam B, the light illumination time points, the duration of light illumination each time, the frequency of light illumination, and the total course of light illumination of the user U based on the measurement result R of the physiological monitoring apparatus 140.

For example, the controller 130 presets a plurality of light source recipes corresponding to different measurement results R, and the controller 130 selects the optimal light source recipe based on the measurement result R of the physiological monitoring apparatus 140. In an embodiment, the doctor remotely monitors the physiological monitoring apparatus 140, and then remotely controls the controller 130 according to the measurement result R to cause the light source 110 to provide the light beam B of the optimal light source recipe. In this framework, if the light source apparatus 300 is a portable light source apparatus, the user U may undergo light treatment or light health care wherever he or she is. On the other hand, if the light source apparatus 300 is in the form of a fixed light fixture, it is up to the user U to select the location for placing the light source apparatus 300, and it is possible to undergo light treatment or light health care without going to a clinic or hospital.

In summary of the above, in the light source apparatus of the embodiments of the disclosure, the optimal light source recipe, the implementation procedure, and the light health care and light treatment product and system corresponding to different purposes of light source application are designed by taking into account the vision, the psychology, the physiological effect, and the biological effect of the human body, and the effect of adjustment of the emotional state, health care, or treatment is thereby achieved.

Although the embodiments are already disclosed as above, these embodiments should not be construed as limitations on the scope of the disclosure. It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A light source apparatus comprising:
a light source emitting a light beam, wherein the light beam illuminates a user so that at least one brain wave index of at least one region in frontal lobe regions of the user changes.

2. The light source apparatus according to claim 1, wherein an irradiance of at least one of a blue light and a green light in the light beam entering eyes of the user falls in a range of 30 μW/cm² to 200 μW/cm².

3. The light source apparatus according to claim 1, wherein a p-value of a statistical test on a change in the at least one brain wave index is smaller than 0.05.

4. The light source apparatus according to claim 3, wherein a color temperature of the light beam falls in a range of 4500 K to 6500 K, an illuminance of the light beam falls in a range of 700 lux to 3000 lux, and a color rendering index of the light beam is greater than or equal to 70.

5. The light source apparatus according to claim 4, wherein an irradiance of a blue light in the light beam entering eyes of the user falls in a range of 30 μW/cm² to 200 μW/cm².

6. The light source apparatus according to claim 5, wherein the at least one brain wave index comprises an α wave power, and the change in the at least one brain wave index of the at least one region satisfies:
Ln(F4)−Ln(F3)>0, Ln(F8)−Ln(F7)>0, or Ln(Fp2)−Ln(Fp1)>0, wherein Ln(F4) is a natural logarithm of an α wave or θ wave power of a first region in the frontal lobe regions of the user, Ln(F3) is a natural logarithm of the α wave or θ wave power of a second region in the frontal lobe regions of the user, Ln(F8) is a natural logarithm of the α wave or θ wave power of the first region in the frontal lobe regions of the user, Ln(F7) is a natural logarithm of the α wave or θ wave power of the second region in the frontal lobe regions of the user, Ln(Fp2) is a natural logarithm of the α wave or θ wave power of the first region in the frontal lobe regions of the user, and Ln(Fp1) is a natural logarithm of the α wave or θ wave power of the second region in the frontal lobe regions of the user.

7. The light source apparatus according to claim 5, wherein an irradiance of a green light in the light beam entering eyes of the user falls in a range of 30 μW/cm² to 200 μW/cm², and the irradiance of the green light is greater than the irradiance of the blue light.

8. The light source apparatus according to claim 7, wherein the at least one brain wave index comprises at least one of a γwave amplitude and a ratio of a β wave amplitude and an α wave amplitude.

9. The light source apparatus according to claim 3, wherein a color temperature of the light beam falls in a range of 3000 K to 4500 K, an illuminance of the light beam falls in a range of 400 lux to 800 lux, a color rendering index of the light beam is greater than or equal to 70, and an irradiance of a green light in the light beam entering eyes of the user falls in a range of 30 μW/cm² to 200 μW/cm².

10. The light source apparatus according to claim 9, wherein the at least one brain wave index comprises at least one of a low β wave amplitude, a θ wave amplitude, and an α wave amplitude.

11. The light source apparatus according to claim 3, wherein a color temperature of the light beam falls in a range of 3000 K to 4500 K, an illuminance of the light beam is smaller than or equal to 600 lux, a color rendering index of the light beam is greater than or equal to 70, and an irradiance of a green light in the light beam entering eyes of the user falls in a range of 30 μW/cm² to 200 μW/cm².

12. The light source apparatus according to claim 11, wherein the at least one brain wave index comprises a θ wave amplitude.

13. The light source apparatus according to claim 1, wherein the light source comprises at least one red light emitting element, at least one green light emitting element, and at least one blue light emitting element.

14. The light source apparatus according to claim 1, further comprising:
a controller coupled to the light source, wherein the controller changes at least one of a color temperature, an illuminance, a color rendering index of the light beam, irradiances of different color lights in the light beam, light illumination time points, a duration of light illumination each time, a frequency of light illumination, and a total course of light illumination of the user.

15. The light source apparatus according to claim 14, further comprising:
a physiological monitoring apparatus monitoring a state of the user, wherein the physiological monitoring apparatus is coupled to the controller, and the controller changes at least one of the color temperature, the illuminance, the color rendering index of the light beam, the irradiances of different color lights in the light beam, the light illumination time points, the duration of light illumination each time, the frequency of light illumination, and the total course of light illumination of the user based on a measurement result of the physiological monitoring apparatus.

16. A wearable apparatus comprising:
a light source apparatus comprising a light source, wherein the light source emits a light beam, and the light beam illuminates a user so that at least one brain wave index of at least one region in frontal lobe regions of the user changes; and
a fixing member, wherein the light source apparatus is disposed on the fixing member and is located around eyes of the user.

17. The wearable apparatus according to claim 16, wherein the light source apparatus comprises an optical filter module.
